(19) United States
(12) Patent Application Publication
(10) Pub. No.: US 2007/0105158 A1
(43) Pub. Date: May 10, 2007

(54) Recombined cell system for deorphanizing g protein-coupled receptors

(75) Inventors: Dietmar Krautwurst, Nuthetal (DE); Elena Shirokova, Potsdam (DE); Kristin Schmiedeberg, Potsdam (DE); Klaus Willecke, Konigswinter (DE); Heiner Niessen, Koln (DE); Peter Bedner, Koln (DE); Jan-Dirk Raguse, Berlin (DE); Wolfgang Meyerhof, Nordestedt (DE)

Correspondence Address: NORRIS, MCLAUGHLIN & MARCUS, 875 THIRD AVE, 18TH FLOOR, NEW YORK, NY, 10022, US

(73) Assignee: Deutsches Institut fur Ernahrungsforschung-Stiftun, Bergholz-Rehbrucke (DE)

(21) Appl. No.: 10/577,094

(22) PCT Filed: Oct. 26, 2004
(86) PCT No.: PCT/EP04/12086

§ 371 Date: Oct. 13, 2006

(30) Foreign Application Priority Data
Oct. 27, 2003 (DE) 103 50 054.5
Apr. 20, 2004 (DE) 10 2004 019 028.3

(51) Int. Cl. G01N 33/567; C07H 21/04; C12P 21/06; C07K 14/705

(52) U.S. Cl. 435/7.2; 435/69.1; 435/320.1; 435/325; 530/350; 536/23.5

(57) ABSTRACT

The present invention relates to a recombinant cellular system, comprising an animal host cell, comprising a recombinant G protein-coupled specific receptor, and the recombinant Ca2+ specific channel CNGA2. The invention furthermore relates to a method for producing the cellular system according to the invention and the use of the system for a deorphanisation of G protein-coupled receptors. Furthermore, the present invention relates to the use of the cellular system for identifying novel G protein-coupled receptors from gene banks.

Recombinant cellular system for the deorphanisation of G protein-coupled receptors The present invention relates to a recombinant cellular system, comprising an animal host cell, comprising a recombinant G protein-coupled specific receptor, and the recombinant Ca2+ specific channel CNGA2. The invention furthermore relates to a method for producing the cellular system according to the invention, and the use of the system for the deorphanisation of G protein-coupled receptors. Furthermore, the present invention relates to the use of the cellular system for identifying novel G protein-coupled receptors from gene banks.

### STATE OF THE ART

The superfamily of G protein-coupled receptors (GPCRs; 7TMs) is one of the largest families of genes identified by man, and has a proven history as an excellent source of drug targets. They react on a large number of stimuli, including small peptides, lipid analogs, amino acid-derivatives and sensory stimuli, such as, for example, light, taste, and smell, and transfer signals into the inner of the cell by interaction with (amongst others) heterotrimeric G proteins. The nearly complete sequencing of the human genome allowed for the identification of a large number of sequences that encode for the so-called “orphan” GPCRs, potential receptors whose natural ligands yet have to be identified. In many cases, the extent of the sequence homology with known receptors is not sufficient in order to find the natural ligand for these orphan receptors, although it is usually possible to determine the possible nature of the respective ligand, such as, for example, a peptide, lipid, nucleotide etc. The so-called “deorphanisation” of these novel GPCRs and the determining of their biological functions has developed into a major aim of many of the large pharmaceutical companies as well as several academic groups. Since 1995, more than 50 ligands for orphan GPCRs were discovered through the use of the orphan receptors as a biosensor and screening for candidate-compounds, wherein it was looked at a biological response (the so-called “reverse pharmacology” approach) (Szekeres P G. Functional assays for identifying ligands at orphan G protein-coupled receptors. Receptors Channels. 2002; 8(5-6):297-308).

Briefly, the reverse molecular pharmacological technique includes the cloning and the expression of orphan GPCRs in mammalian cells, and screening in these cells for a functional response against cognate or surrogate agonists that are present in biological extract preparations, peptide-libraries, and complex collections of compounds. The functional genomics approach includes the use of “humanised” yeast cells, whereby the yeast cell-GPCR transduction system is modified in such a way to allow for a functional expression and coupling of human GPCRs to the endogenous signal machinery. Both systems provide an excellent platform for the identification of novel receptor ligands. As soon as activating ligands are identified, these can be used as pharmacological tools in order to examine the receptor function and the relation to diseases, including obesity, inflammatory diseases, heart diseases, and cancer. (Wilson S, Bergsma D J, Chambers J K, Muir A I, Fantom K G, Ellis C, Murdock P R, Herrity N C, Stadel J M. Orphan G-protein-coupled receptors: the next generation of drug targets? Br J. Pharmacol. 1998 December; 125(7):1387-92; Shaaban S, Benton B. Orphan G protein-coupled receptors: from DNA to drug targets. Curr Opin Drug Discov Devel. 2001 September; 4(5):535-47).

A further approach for identifying receptor ligands is the comparison of known and/or putative GPCRs that are available in the database (Joost P, Methner A. Phylogenetic analysis of 277 human G-protein-coupled receptors as a tool for the prediction of orphan receptor ligands. Genome Biol. 2002 Oct. 17; 3(11):RESEARCH0063). This comparison can also be made between different species in order to identify receptors that, for example, have been identified in the mouse (Vassilatis D K, Hohmann J G, Zeng H, Li F, Ranchalis J E, Mortrud M T, Brown A, Rodriguez S S, Weller J R, Wright A C, Bergmann J E, Gaitanaris G A. The G protein-coupled receptor repertoires of human and mouse. Proc Natl Acad Sci USA. 2003 Apr. 15; 100(8):4903-8. Epub 2003 Apr. 4).

In addition to the groups of particular guanylyl-cyclases (reviewed in, e.g., Lucas K A, et al. guanylyl cyclases and signalling by cyclic GMP. Pharmacol Rev. 2000 September; 52(3):375-414, Gibson A D, Garbers D L. Guanylyl cyclases as a family of putative odorant receptors. Annu Rev Neurosci. 2000; 23:417-39), pheromone receptors, e.g. of the V1R-type (reviewed in, e.g., Matsunami H, Amrein H. Taste and pheromone perception in mammals and flies. Genome Biol. 2003; 4(7):220. Epub 2003 Jun. 30; Dulac C, Torello A T. Molecular detection of pheromone signals in mammals: from genes to behaviour. Nat Rev Neurosci. 2003 July; 4(7):551-62), and the adrenalin receptors, e.g. the alpha- or beta-adrenergic receptors (reviewed in, e.g., Koshimizu T A, et al. Recent progress in alpha 1-adrenoceptor pharmacology. Biol Pharm Bull. 2002 April; 25(4):401-8), which differ by different intracellular signalling cascades, a group which is of particular interest is the one of the olfactory receptors, for which already a large number of patent applications have been filed (e.g. EP 1301599; CN1386760; CN1380323; CN1376713; CN1376691; AU2147402; EP1235859, and WO 02/059274).

The olfactory system enables the vertebrates to detect a large number of chemically different odorant molecules, and to distinguish them one from the other. In the human and the mouse, 350 to 1000 odorant receptor (OR) genes each encode for seven transmembrane-spanning (7TM) and G protein-coupled receptors (GPCR), respectively (Buck and Axel, 1991; Zozulya et al., 2001; Zhang and Firestein, 2002) that are expressed in the olfactory sensory neurons (OSN) of the olfactory epithelium (OE) (for a review, see Mombaerts, 1999; Young and Trask, 2002). A particular OSN most likely expresses only a single type of OR (Chess et al., 1994; Malnic et al., 1999), and individual OSNs often show a broadly adjusted odorant specificity that partially depends from the odorant-concentration (Sicard and Holley, 1984; Sato et al., 1994; Malnic et al., 1999; Duchamp-Viret et al., 2000; Ma and Shepherd, 2000; Hamana et al., 2003). Thus, the odorant-distinguishing-, quality- and intensity coding of each particular OSN depends from the EC50 odorant profile of the particular OR type, expressing it (Malnic et al., 1999; Kajiya et al., 2001; Hamana et al., 2003). More than four spatially distinct expression regions of the OR gene were described in the OE of mice (for a review, see Touhara, 2002). A systematic distribution of the odorant sensitivity above the OE were shown by elektro-olfactogram (EOG)-recordings and in situ Ca2+ imaging (Scott and Brierley, 1999; Omura et al., 2003). Nevertheless, until today, information about the molecular determinants that are the basis of each spatially organised odorant-response zone, e.g. odorant recognition profiles of OR and their zonal expression profiles within the OE are lacking.

Subsequent to odorant stimulation, the OR activates an olfactory-specific signal transduction-cascade (Reed, 1992; Gold, 1999) which includes the G-protein G-alpha-olf (Jones and Reed, 1989; Firestein et al., 1991), the adenylate cyclase (AC) type III (Bakalyar and Reed, 1990), and a heteromeric cyclic nucleotide-gating (CNG) Ca2+-permeable cationic channel (Nakamura and Gold, 1987; Dhallan et al., 1990; Dzeija et al., 1999). Consistently, gene-targeting deletions of G-alpha-olf, ACE, or the CNGA2 cannel-subunit (Brunet et al., 1996; Belluscio et al., 1998; Baker et al., 1999; Wong et al., 2000) rendered mice largely anosmic. Another olfactory signal transduction signalling pathway which is specifically modulated by cGMP includes the particular guanylyl cyclase type D (GC-D) that is present in a sub-group of OSN (Juilfs et al., 1997; Meyer et al., 2000). Despite the precise knowledge about the genetics of OR and their signal transduction components, only a limited number of studies brought odorants in relation with individual OR, by means of adenoviral over-expression assays in vivo (Zhao et al., 1998; Araneda et al., 2000), heterologous expression of recombinant OR in vitro (Krautwurst et al., 1998; Wetzel et al., 1999; Gaillard et al., 2002), single cell RT-PCR of odorant-responsive OSN (Malnic et al., 1999; Touhara et al., 1999; Kajiya et al., 2001), and OR gene-targeting, followed by functional analysis of single-OSN (Bozza et al., 2002). The functional expression of some N-terminally-labelled OR in HEK-293 cells were achieved through artificial coupling of these to phosphoinositol-signalling and calcium release via the G-protein subunits alpha15,16 (Krautwurst et al., 1998; Touhara et al., 1999; Kajiya et al., 2001; Gaillard et al., 2002) or via G-alpha-q/11 (Wetzel et al., 1999). Nevertheless, it is not known, whether all OR, and with what efficiency, can couple via the G-proteins alpha15,16. Recent re-examination of results of other groups have shown that the alpha15,16 system led to some false-negative results.

The problems that have been found during the examination of the receptors of the olfactory system can be transferred to the other classes of receptors as mentioned above. Thus, until today, a cellular system is lacking in order to functionally screen and characterise GPCR in a suitable genetic background, and in particular ORs in their original G-alpha-olf-ACIII-cAMP signalling-background. At the same time, a cellular system with a suitable genetic background is lacking, by means of which novel orphan-receptors can be quickly found and characterised in a large scale.

It is an object of the present invention to provide a cellular system in order to broaden and improve the spectrum of methods for identifying and characterising (deorphanisation) of receptors and their respective ligands. The system should optimally be producible with low costs, largely allow for established measuring techniques, and, in addition, have a low genetic safety level. In addition, false results should be excluded as much as possible.

According to a first aspect of the present invention, this object of the present invention is solved by a recombinant cellular system, wherein the system comprises an animal host cell, comprising the following recombinant proteins; A) a recombinant specific G protein-coupled receptor, and B) the recombinant Ca2+ permeable channel CNGA2. Further preferred embodiments of the system according to the invention are claimed in the dependent claims. According to the present invention, the recombinant Ca2+ specific channel CNGA2 can be composed as a homomer or heteromer. Preferred is a subunit-homomer.

The present invention in part relies on the finding, that in one, case the heterologous expression of an OR was associated with odorant-induced cAMP production (Kajiya et al., 2001), suggesting that a recombinant OR couples to endogenous G-alpha-s and AC proteins in a human cell line.

WO 03/004611 describes the expression of the functional human olfactory nucleotide-gating (CNG) channel subunit OCNC1 in recombinant host cells and its use in cell-based tests, in order to identify odorant modulators. Tests are performed with the channel itself. U.S. Pat. No. 6,492,143 describes olfactory receptor expression libraries and methods for their production and use. WO 01/51609 then describes the isolation and in vitro differentiation of conditionally immortalised mouse-olfactory receptor neurons. Therefore, none of the above indicated publications discloses or proposes a cellular system according to the invention.

Preferred is a recombinant cellular system according to the present invention that furthermore comprises a recombinant protein from the group of connexins, e.g. Cx43 or Cx26. By introducing a connexion, the sensitivity of the system according to the invention is improved, since Ca2+ can be spread between the cells.

A particularly preferred recombinant cellular system according to the invention comprises a recombinant specific G protein-coupled receptor, that is selected from the group of the particular guanylyl-cyclases, e.g. type A to G. Thus, the components of the recombinant cellular system according to the invention in this case consist of A) a recombinant specific G protein-coupled receptor from the group of the particular guanylyl-cyclases, and B) the recombinant Ca2+ specific channel CNGA2. Optionally, also a connexin can be present.

A further aspect of the present invention then relates to a recombinant cellular system according to the invention which furthermore comprises a cyclase that is harmonised with the specific G protein-coupled receptor, e.g. an adenylyl- or guanylyl-cyclase. The components of the recombinant cellular system according to the invention in this case consist of A) a recombinant specific G protein-coupled receptor, B) the recombinant Ca2+ specific channel CNGA2 and C) the cyclase that is harmonised with the specific receptor. Optionally, also here a connexin can be present. Preferred is a recombinant cellular system according to the invention, wherein the recombinant specific G protein-coupled receptor is selected from the group of pheromone receptors, e.g. of the V1R-type with all families VR-a to VR-1, including the V3R-type (VR-d), for example V1R-b2, the hormone receptors, e.g. the beta-adrenergic receptors, and the olfactory receptors, e.g. OR1A1, OR1A2, Olfr43, Olfr49, MOR261-10, MOR267-1, LOC331758, Olfr41, or Olfr6.

In the context of the examinations for the present invention, the inventors have furthermore found that the sensitivity of the cellular system substantially increases, when additionally, a recombinant G-protein is present that is harmonised with the specific G protein-coupled receptor, e.g. G-alpha-olf. Thus, the introduction of such a G-protein is therefore preferred according to the invention. Upon introduction of a corresponding G-protein, conveniently, a priming of the cellular system in most cases is not required, which simplifies the measuring.

A further aspect of the present invention relates to a recombinant cellular system, wherein the animal host cell is selected from murine cell lines or human cell lines, e.g. human cancer cell lines, such as, for example, HeLa or HEK293. Many cell lines can be used, it is only important that the corresponding genetic background is selected in such a manner that the corresponding signalling cascade is present. The person of skill will be readily able to realize, which cell lines are suitable.

Finally, particularly preferred recombinant cellular system according to the invention is selected from the cellular systems

HeLa-Cx43/CNGA2/Olfr49;

HeLa-Cx43/CNGA2/G-alpha-olf;

HeLa-Cx43/CNGA2/G-alpha-olf/Olfr 49;

HeLa-Cx43/CNGA2/G-alpha-olf/Olfr41;

HeLa-Cx43/CNGA2/G-alpha-olf/Olfr 6 or

HeLa-Cx43/CNGA2/G-alpha-olf/OR1A1.

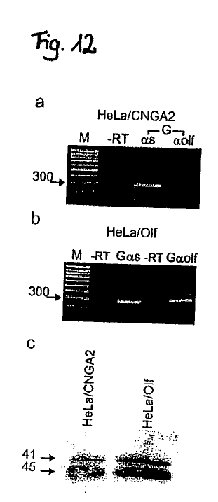

A particularly preferred recombinant cell line “HeLa/olf” according to the invention with the components HeLa-Cx43 (from the rat)/CNGA2 (bovine)/G-alpha-olf (from the human, over-expressed, see FIG. 12c) was deposited on Apr. 20, 2004 at the DSMZ—Deutsche Sammlung von Mikroorganismen and Zellkulturen GmbH in Mascheroder Weg 1b, D-38124 Braunschweig. The deposit has obtained the number DSM ACC2649.

A further particularly preferred recombinant cellular system according to the invention is characterised in that the recombinant proteins are present stable, over-expressed and/or transiently transfected. “Stable” in the context of the present invention shall mean an expression for over at least 10 to 13 passages at 1 to 2 passages per week (for this, see also the corresponding example below). This stable transfection was not present in common systems, and therefore represents another advantage of the system according to the invention. In the context of the present invention, “over-expressed” shall mean an amplification of the expression of G-proteins in the cell above the natural extent. On the one hand, this comprises an additive increase of the G-protein-expression, as well as an increased expression of one (in case of HeLa-Olf both) G-protein(s) (for this, see e.g. FIG. 12c). In case of the HeLa-Olf-cells, the over-expression is most likely caused by the CMV-promoter as used.

A further aspect of the present invention relates to a method for identifying receptor activating substances, comprising the steps of a) providing a recombinant cellular system according to the invention, b) contacting of the cellular system with a potential G protein-coupled receptor-inducing substance, and c) measuring of the activation or inhibition of the Ca2+ influx into the cell. Such screening methods can be easily designed by the person of skill on the basis of the methods as described here, and the extensive literature in the field of screening (e.g. Szekeres P G. Functional assays for identifying ligands at orphan G protein-coupled receptors. Receptors Channels. 2002; 8(5-6):297-308). Particularly preferred the method is performed in high-throughput. Further preferred is the screening with substances that are already known to be odorants, such as, for example, (−)citronellal or beta-citronellol, pheromones, hormones, such as, for example, adrenalin, or natriuretic peptide type-C.

The measuring method that is used according to the invention for measuring the activation or inhibition of the Ca2+ influx into the cell can be any suitable method, nevertheless, preferred is a method according to the invention which includes a loading of the cell with Fura-2-AM or Fluo-4-AM, and measuring of the emission-wavelength at 515 nm. In some cases, it can be reasonable that the cellular system is pre-treated for the measurement with an enhancer, such as, for example, forskolin or thapsigargin.

As mentioned above, newly identified G protein-coupled receptor inducing-substances can constitute the basis for valuable pharmaceutical products or “lead-structures” for the development of such pharmaceutical products. In a further aspect, the present invention therefore relates to a method for producing a pharmaceutical composition, comprising the steps of a) performing a method for identifying receptor activating-substances according to the present invention, and b) formulating of the obtained G protein-coupled receptor inducing substance with known auxiliary agents and additives. The actual formulation poses no problem for the person of skill, depends from each of the substances to be formulated, and can be readily taken from the respective literature.

A further aspect of the present invention relates to a recombinant cellular system according to the invention, wherein the cellular system comprises a potential recombinant specific G protein-coupled receptor instead of an already known or orphan-receptor. Thus, the cellular system according to the invention can serve as a basis for the identification of further orphan G protein coupled receptors, in that expressed genes are introduced as a cassette into the cellular system, and their ability to trigger an activation or inhibition of the Ca2+ influx in response to certain stimuli (e.g. odorants) is analysed. Suitable sources of such expressed genes are commercially available animal and/or tissue-specific banks, which, for example, can comprise the proteome of a cell. Also in this case, it is preferred that the method takes place in a high-throughput environment, e.g. in microtiter-plates in a fluorescence-plate reader, or high-resolution microscopy-supported on the level of individual cells.

Thus, the invention furthermore relates to a method for identifying of novel G protein-coupled receptors, comprising the steps of a) providing a suitable recombinant cellular system according to the invention as above, b) contacting of the cellular system with a receptor-activating substance or presumably G protein-coupled receptor-activating substance, and c) measuring of the activation or inhibition of the Ca2+ influx into the cell.

A further aspect of the present invention then relates to a method for producing a recombinant cellular system, comprising a) providing of a suitable animal host cell, b) introducing a recombinant specific G protein-coupled receptor or a potential recombinant specific G protein-coupled receptor, and c) introducing the recombinant Ca2+ permeable channel CNGA2. According to the invention, the recombinant Ca2+ permeable channel CNGA2 can be introduced as a homomer or heteromer. Preferred is a subunit-homomer.

Preferred is a method according to the invention that furthermore comprises introducing of a recombinant protein from the group of the connexins, e.g. Cx43 or Cx26. By introducing of the connexion, the sensitivity of the system according to the invention is improved, since Ca2+ can be dispersed between the cells. Further preferred is a method according to the invention which comprises introducing of a cyclase that is harmonised with the specific G protein-coupled receptor, e.g. an adenylyl- or guanylyl-cyclase, and/or introducing of a recombinant G-protein that is harmonised with the specific G protein-coupled receptor, e.g. G-alpha-olf.

According to the invention, any technique for the introduction of the genetic constructs known to the person of skill can be used. Preferred according to the invention is a method, wherein the introducing is selected from transfection, e.g. Ca2+-phosphate-transfection, lipofection, and transduction, as well as subsequent optional integration into the genome with the aid of a recombinase and/or antibiotic-selection cloning, and transduction.

As already mentioned above, the recombinant cellular system according to the invention can be used for a deorphanisation of G protein-coupled receptors through identifying of corresponding G protein-coupled receptor inducing substances, e.g. odorants. At the same time, the cellular system according to the invention can also be used for identifying novel cellular G protein-coupled orphan-receptors. These receptors can even be identified and deorphanised in a single run-through, if the substance that is used for screening is simultaneously identified as the substance that is specific for the receptor.

The inventors now have stably reconstituted the olfactory signal transduction in HeLa/Olf cells, from the olfactory receptors via the G-protein alpha-olf and the adenylyl cyclases type III to the homomeric olfactory cyclic nucleotide-gating CNGA2 channel. The signalling efficiency of the olfactory receptors in HeLa/Olf cells was increased by the presence of G-alpha-olf, compared to their signalling via endogenous G-alpha-s. The CNGA2 channel functions as a sensor that indicates changes in the intracellular cyclic nucleotide-concentration through a calcium-influx that can be followed by fluorescence imaging techniques.

Reconstitution of olfactory receptor-cAMP-signalling in HeLa cells.—In order to deorphanise olfactory receptors by a functional genomics approach, the inventor's transfected a diverse olfactory receptor sub-genome into HeLa/Olf cells. The inventors identified novel 3 of 93 (red, yellow, green; control: purple) mouse rhodopsin-labelled olfactory receptor chimeras which, in a 96-well screening approach, reacted differently to 1 μM (−)citronellal or 10 μM beta-citronellol.

Nevertheless, functional genomics of OR was largely obstructed by the lack of a cellular system in order to examine recombinant ORs in their olfactory signalling background. Here, the inventors for the first time have described the functional reconstitution of a partial OR genome, together with its olfactory signal transduction molecules, in a human cell line, in order to examine the odorant-coding on the level of the receptor.

Cyclic nucleotide-sensing and Ca2+ signalling via CNGA2—The experiments as shown here with HeLa-Cx43/CNGA2 cells demonstrate their utility for the functional screening, the deorphanisation, and characterisation of non-olfactory GPCR or particular GC that are involved in the cAMP or cGMP signalling. The EC50 values which the inventors determined in HeLa-Cx43/CNGA2 cells for isoproterenol that acts on endogenous P-AR, and CNP that acts on recombinant GC-B, are in agreement with the literature (Lucas et al., 2000; Crider and Sharif, 2002). Our determinations of the cAMP production in these cells was used to link the ligand-dependent stimulation of receptor/G protein combinations to the function of the CNGA2 channel as a cyclic nucleotide-dependent Ca2+ influx reporter. The inventors have shown a strong correlation between the use of the agonists of the stimulation of Ca2+ influx, by showing that ligands, at their EC50, produce an intracellular cAMP concentration which is close to their EC50 for the olfactory homomeric CNGA2 channel.

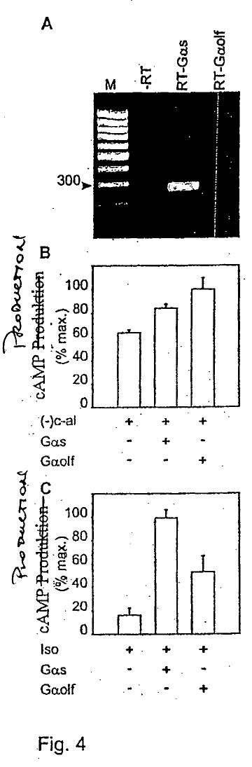

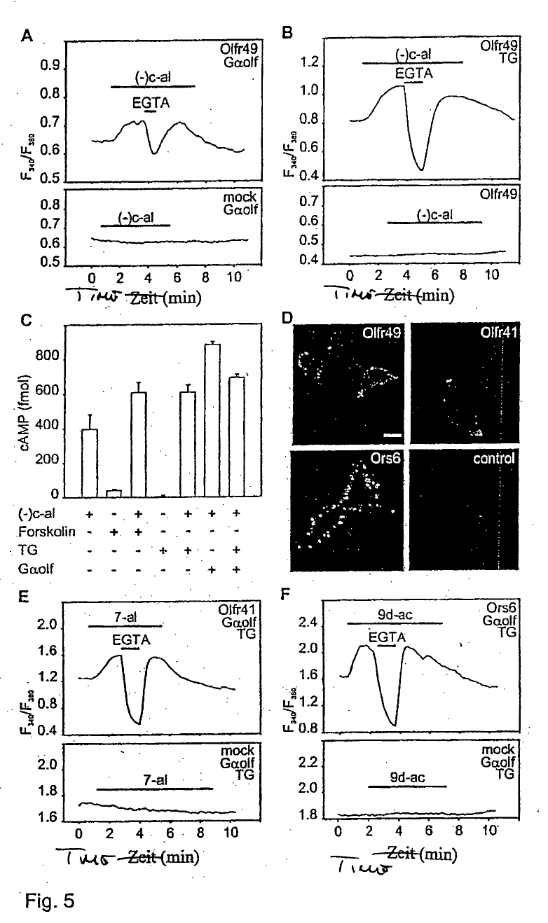

Olfactory signal transduction and preferential coupling of OR via G-alpha-olf in HeLa cells. In both Ca2+ imaging-experiments and cAMP assays with HeLa-Cx43/CNGA2 cells, the inventors could show that the signalling efficiency of OR via the cAMP signalling pathway is improved by the pre-treatment (priming) with either forskolin or thapsigargin. Nevertheless, the inventors also show that the enriching of HeLa-Cx43/CNGA2 cells with G protein increased the signalling efficiency of receptors, thereby removing the requirement of a pre-treatment with forskolin or thapsigargin. Thus, it appears to be important to express a sufficient amount of signal transducing components, the (−)citronellal- or isoproterenol-induced cAMP production was readily observed in HeLa-Cx43/CNGA2/rho-tag(39)-Olfr49 cells, even without a pre-treatment with forskolin, thapsigargin or over-expression of G-alpha-olf. This is most likely due to the coupling of receptors via endogenous by expressed G-alpha-s. The cAMP tests furthermore showed a coupling of Olfr49 or beta-AR to either G-alpha-olf and G-alpha-s, and, in addition, showed a preferred coupling of Olfr49 or beta-AR to G-alpha-olf or G-alpha-s, respectively, whereby earlier results have been confirmed and extended (Jones et al., 1990; Kajiya et al., 2001; Liu et al., 2001). The smaller difference in the OR-induced cAMP production of G-alpha-olf, compared to G-alpha-s enriched HeLa-Cx43/CNGA2/rho-tag(39)-Olfr49 cells, compared to the differences in the beta-AR-induced cAMP levels (FIG. 4) can be explained by a faster deactivation of G-alpha-olf-GTP through the GTP hydrolysis and the GTP dissociation relative to G-alpha-s-GTP (Liu et al., 2001). The thapsigargin-induced reduction in the (−)citronellal-stimulated cAMP production that was observed in HeLa-Cx43/CNGA2/rho-tag(39)-Olfr49 cells that express hG-alpha-olf (FIG. 5), can be explained by an inhibition of ACVI due to an increased intracellular Ca2+ concentration.

Functional genomics with a OR sub-genome—Effects of the odorant concentration. On the level of the OR-ligand interaction, a combinatory coding is commonly accepted, as derived from often broadly “tuned” OSNs. The assumption is that one OR can be activated by different odorants, and that one odorant can activate several ORs with different efficiencies. Nevertheless, until now only 14 OR gene sequences were published in one (Malnic et al., 1999) but not the other (Hamana et al., 2003) of two comparative examinations which tried to derive OR sequences from hundreds of single cell RT-PCR experiments with mouse OSN that had reacted to a homologous set of odorants. In none of these studies, the derived OR sequences were used in order to confirm the odorant responsivity of their gene products, or to characterise their EC50-ranking odorant profile by means of functional heterologous expression. For one of the derived putative ORs, Ors6 (Malnic et al., 1999), the inventors have now shown an odorant responsivity in HeLa-Cx43/CNGA2/hG-alpha-olf cells (FIG. 5) similar to those in OSN. In another study, a library of recombinant OR chimeras was expressed in HEK-293, and functionally screened with odorants (Krautwurst et al., 1998). From these experiments, Olfr49 resulted as a responder for (−)citronellal, which now was stably expressed by the inventors and characterised by its EC50-ranking odorant profile (FIG. 4).

In the present invention, the inventors have now studied the aspect that the odorant/receptor encoding depends from the concentration of the odorant. By a functional genomics approach whereby 93 mouse OR-cell lines were screened against (−)citronellal and beta-citronellol, the inventors newly identified 3 OR that responded to both odorants, or both responded specifically. An increasing number of responding OR (from 3-9% to 22-59%) as a result of an increasing concentration of (−)citronellal suggests a combinatory coding of the odorant quality and/or -intensity by different OR subgroups. This phenomenon, which was initially observed in the Ca2+ imaging or in electro-physiological experiments, until now was only observed with OSNs, whereby 4 to 57% of the OSN responded to increasing micromolar concentrations of particular odorants (Sato et al., 1994; Malnic et al., 1999; Duchamp-Viret et al., 2000; Ma and Shepherd, 2000; Hamana et al., 2003). The number of OSNs, and thus Ors, that recognised different odorants, considerably varied in these examinations depending from the odorants and their concentrations.

The inventors observed a similar variation in their experiments, which is possibly due to the use of different OR subgroups, and partially could also reflect experimental variations, e.g. differences in the transfection efficiency or differences in the plasma membrane-expression. Recently, an advantage for the unspecific versus the specific tuning of OSN, and thus ORs, for the quality and the intensity coding by means of a mixture of receptive fields of an as large as possible diversity was proposed in a mathematical model (Sanchez-Montanes and Pearce, 2002). Nevertheless, a recent study suggests that the principal odour qualities are encoded by the most sensitive receptors for a particular odorant (Hamana et al., 2003).

The human OR1A1, which is the ortholog OR (84%) to mouse Olfr43 and LOC331758, has maintained a similar specificity for (−)citronellal, whereby its concentration-response-ratio starts at about the human threshold-value. On the other hand, Olfr49, and many other Ors, can be regarded as “generalists” for (−)citronellal. The inventors thus have presented evidence that Olfr43 and LOC331758 in the mouse and OR1A1 and OR1A2 in the human are candidates for being specialists of the ORs for the key food odorant (−)citronellal. On the long run, nevertheless, only a holistic approach, for example a screening of all human ORs (Zozulya et al., 2001), and an establishing of EC50-based odorant profiles for all responding ORs, can lead to a complete picture with respect to the question, which OR, or which subgroup of ORs, represents the odour quality of, e.g., citronellal.

The results as described above were obtained and discussed in relation to the analysis of a particular odorant ((−)citronellal) and its respective receptor. The results as described, nevertheless, can be readily extended to other receptor-families, without departing from the scope of the present invention. By using the results as described herein, and the following examples, the person of skill can readily adapt the method of the present invention in a suitable manner in order to also examine and/or identify additional G protein coupled receptors of other classes. Therefore, the cellular system according to the invention can be universally employed within the G protein coupled receptors.

The invention shall now be further illustrated by the attached examples with reference to the attached Figures, nevertheless, without being limited thereto.

In the Figures:

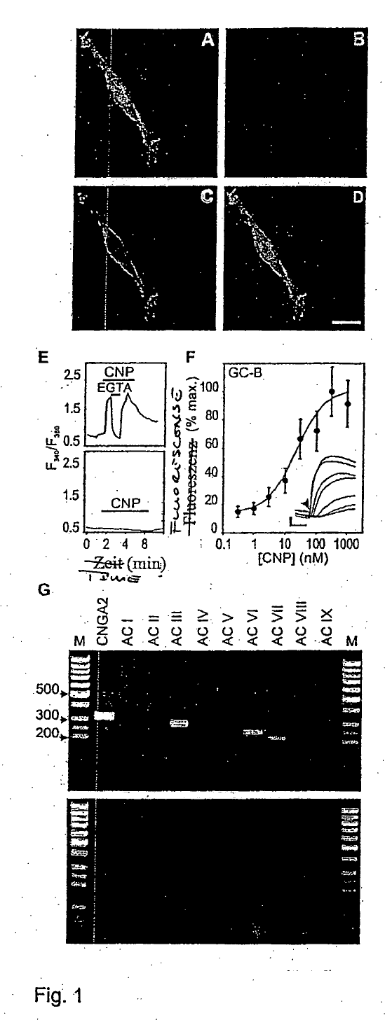

FIG. 1: shows HeLa-Cx43/CNGA2 cells express the CNGA2 channel and the RNA for four endogenous adenylyl cyclases. (A-D) confocal fluorescence images of HeLa-Cx43/CNGA2 cells. (A) Permeabilised, anti-CNGA2/Alexa-488-labelled cell. (B) Primary antibodies omitted. (C) The cellular surface is made visible with concanavalin A/Texas Red. (D) Overlay of (A) and (C), with co-localised signals in yellow. Scale bars, 20 μm. (E) CNP induced a Ca2+ influx into IBMX-pre-treated cells, transfected with DNA for GC-B. Lower panel, control-transfected cells. Mean measurements of 6 responders/25 total-cells, and all cells in the control. (F) Concentration-response ratio of CNP. The data are mean values±SD from 5-well-determinations, EC50=0.027 μM. Insertion, fluorescence measurements. Arrowhead, use of CNP. Vertical scale, 200 fluorescence counts; horizontal scale, 1 min. (G) RT-PCR products of HeLa-Cx43/CNGA2 mRNA, by using gene-specific primers for the olfactory CNGA2 channel subunit and all known types of human AC (ACI-IX) (upper panel). Lower panel, —RT, wherein reverse transcriptase was omitted. M, marker sizes (base pairs). Similar results were obtained in two independent experiments.

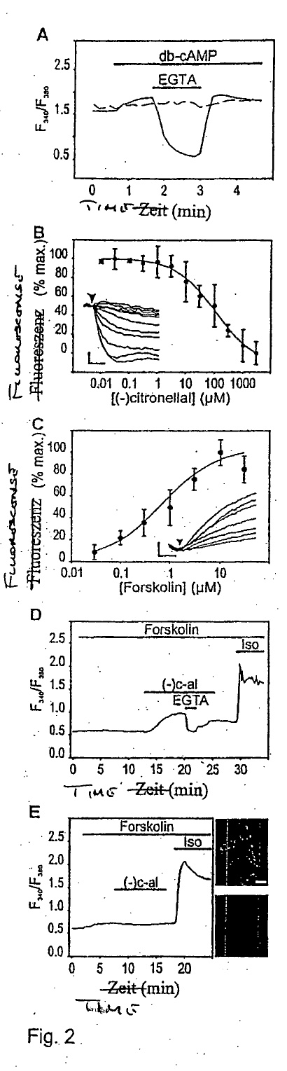

FIG. 2: Shows the tuning of CNGA2 for (−)citronellal/Olfr49-induced Ca2+ influx. (A) Activation of CNGA2 by db-cAMP in thapsigargin- and IBMX-pre-treated HeLa-Cx43/CNGA2 cells, in Ca2+ imaging experiments. Dashed line, cells lacking CNGA2. Mean measurements of all recorded cells. (B) Concentration-response ratio of (−)citronellal in a FLIPR experiment with db-cAMP-pre-treated cells, IC50=107.6 μM. Insertion, fluorescence measurements (0.003-3000 μM, top to bottom). Vertical scale, 200 fluorescence counts; horizontal scale, 30 sec. (C) concentration-response ration of forskolin pre-treatment (EC50=0.70±0.40 μM) in cells, stimulated with (−)Citronellal (10 μM). Insertion, fluorescence measurements below 0.03-30 μM forskolin (from bottom to top, max. effect=10 μM). Vertical scale, 500 fluorescence counts; horizontal scale, 1 min. Data in (B, C) are mean values±SD from 5 well determinations. Arrow heads in insertions, use of (−)citronellal. (D) (−)Citronellal(c-al)-induced Ca2+ influx in forskolin-pre-treated HeLa-Cx43/CNGA2/rho-tag(39)-Olfr49 cells. (E) cells, lacking Olfr49. Mean measurements from all recorded cells. Horizontal bar, bath-application of drugs (10 μM). Iso, isoproterenol. Similar results were obtained in two (A, B) or three (C, D) independent experiments. (E, upper right panel) Confocal images of non-permeabilised B6-30/Alexa-488-labelled HeLa-Cx43/CNGA2/rho-tag(39)-Olfr49 cells. (E, lower right panel), omitting the primary antibody. Scale bar, 16 μm.

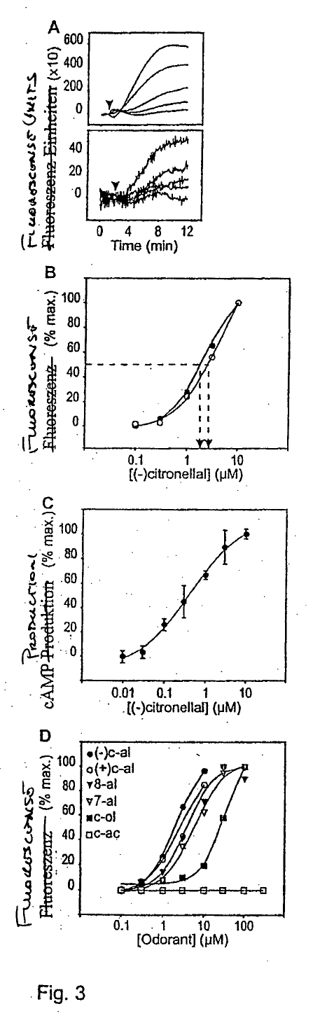

FIG. 3: Odorant specificity and concentration ranges of Olfr49. (A) (−)Citronellal-induced Ca2+ influx in FLIPR experiments with forskolin-pre-treated cells. (A, upper panel) HeLa-Cx43/CNGA2/rho-tag(39)-Olfr49 cells; (A, lower Panel) Note the tenfold lower amplitudes in HeLa-Cx43/CNGA2 cells that were transfected with Olfr49 DNA. (B) Concentration-response ratios with the maximal amplitudes derived from (A, upper panel, filled circles) EC50=2.1±0.07 μM, and (A, lower panel, open circles) EC50=3.9±1.3 μM. (C) (−)Citronellal-induced cAMP production in HeLa-Cx43/CNGA2/rho-tag(39)-Olfr49 cells. The data are mean±SD (n=2 independent experiments), depicted as percent of the maximum (EC50=0.4±0.25 μM). (D) Concentration-response ratio of (−)citronellal ((−)c-al, filled circles), (+)citronellal ((+)c-al, open circles), octanal (8-al, filled triangles), heptanal (7-al, open triangles), beta-citronellol (c-ol, filled squares), and citronellic acid (c-ac, open squares) against odorant-induced Ca2+-influx into forskolin-pre-treated HeLa-Cx43/CNGA2/rho-tag(39)-Olfr49 cells. The data are mean values of n=3 independent experiments, with SD<20%.

FIG. 4: Effects of G-alpha-olf or G-alpha-s over-expression on the receptor/ligand-induced cAMP production. (A) RT-PCR using gene-specific primers on HeLa-Cx43/CNGA2 cDNA. −RT, reverse transcriptase was omitted. M, marker sizes (base pairs). (B, C) HeLa-Cx43/CNGA2/rho-tag(39)-Olfr49 cells, transfected with either G-alpha-olf or G-alpha-s DNA (both rat), IBMX-pre-incubated and stimulated with (−)citronellal (B) or isoproterenol (C), both at 3 μM. Each bar represents the mean±S.D. from three-fold determinations. All differences in the cAMP production are significant at p<0.05 in (B) and (C). Similar results were obtained in two independent experiments.

FIG. 5: Effect of forskolin, thapsigargin and G-alpha-olf on odorant/OR signalling in HeLa-Cx43/CNGA2 cells. (A, E, F) odorant-induced Ca2+ influx in Fura-2-loaded HeLa-Cx43/CNGA2/G-alpha-olf cells, transfected with DNA for rho-tag(39)-Olfr49 (A), −Olfr41 (E), or −Ors6 (F). Lower panel, control-transfected cells. (B) (−)Citronellal-induced Ca2+ influx into Fura-2-loaded HeLa-Cx43/CNGA2/rho-tag(39)-Olfr49 cells without hG-alpha-olf. The concentration of the odorants ((−)c-al, (−)citronellal; 8-al, octanal; 9d-ac, nonandioine acid) was 10 μM. Note that the cells were not treated with forskolin, but with thapsigargin (TG) where indicated. Shown are mean Ca2+ measurements of all responsive cells (8 responders/30 total cells at (A), 4/25 at (E), 12/21 at (F), all cells at (B), and in the control). Similar results were obtained in three independent experiments. (C) cAMP production in IBMX-pre-treated HeLa-Cx43/CNGA2/rho-tag(39)-Olfr49 cells, stimulated with (−)citronellal (10 μM) in the presence of forskolin (2.5 μM), thapsigargin (1 μM) or after transfection with G-alpha-olf (rat). Each bar represents the mean±S.D. from two independent experiments. (D) Confocal pictures of non-permeabilised B6-30/Alexa-488-labelled cells that express rho-tag(39)-OR. Lower right panel, omission of the primary antibody. Scale bars, 16 μm.

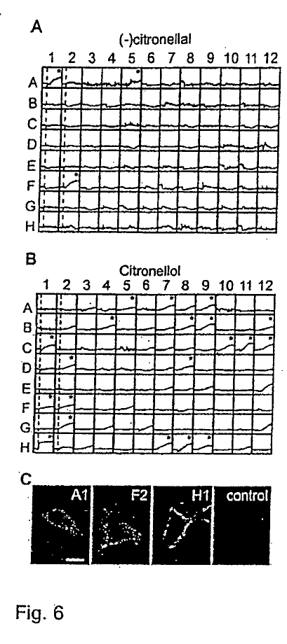

FIG. 6: Screening of 93 OR chimeras with (−)citronellal and beta-citronellol. (A, B) HeLa-Cx43/CNGA2/hG-alpha-olf cells were transfected with DNAs of 93 rho-tag(20)-M4 chimeric mouse ORs, and screened against 1 μM (A), or 10 μM (B) of odorant in FLIPR experiments. Dashed lines, bath-application of odorants. Stars, responder to odorants. The coordinates A10-A12 contained control-transfected cells. Note that the different noise signal of the individual measurements is due to the normalisation of the intrinsic isoproterenol signal amplitude in each experiment. Time scale, 4 min. (C) Confocal pictures of non-permeabilised and B6-30/Alexa-488-labelled cells expressing rho-tag(20)-M4 OR chimeras. Right panel, omission of the primary antibody. Scale bar, 16 μm.

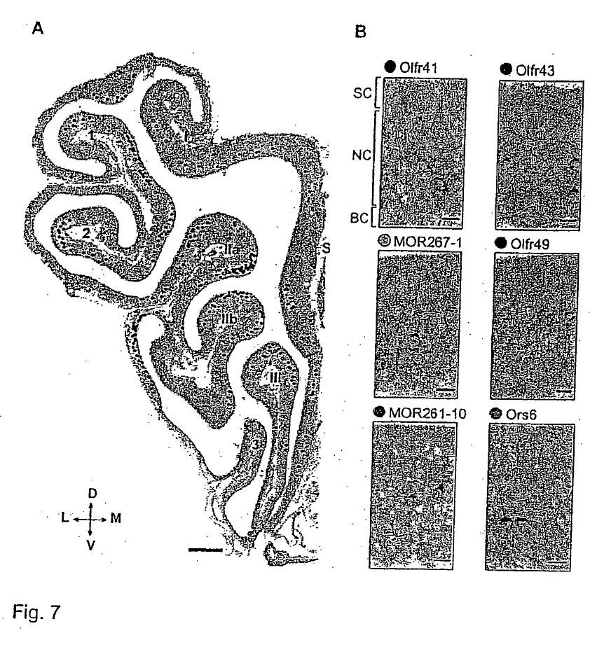

FIG. 7: Differential zonal expression pattern of mouse OR in situ (A) In situ hybridisation of odorant receptor antisense RNA. A coronal half section of mouse OE with ectoturbinates 1-3 and endoturbinates I-III summarizes the expression pattern of ORs. The data are from serial sections, hybridised separately with the respective OR, as depicted by coloured points: Olfr41 (black), Olfr43 (red), MOR267-1 (yellow), Olfr49 (purple), 261-10 (green), and Ors6 (blue). Scale bars, 300 μm. (B) Higher magnification of individual sections of OEs, hybridised with antisense OR-probes. SC, Sustentaculary cells; NC, neuronal cells; BC, basal cells. Scale bar, 10 μm.

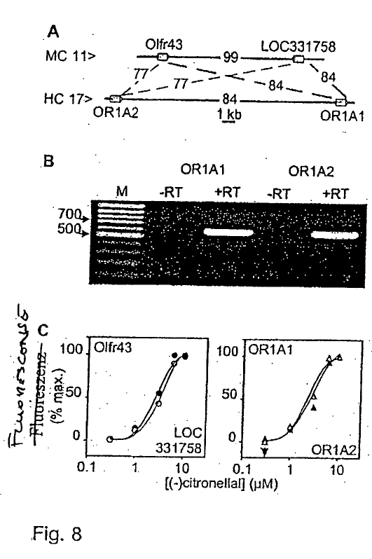

FIG. 8: Gene expression and function of human ORS for (−)citronellal-(A) Candidate-(−)citronellal-receptor gene in synthetic clusters on the mouse-chromosome IIB3-B5 (MC 11), and human chromosome 17p13.3 (HC 17). The arrows show the range and the orientation of the gene, drawn to scale of NCBI mouse and human genomic maps. The numbers indicate the amino acid-identity (%) between the gene products. (B) RT-PCR using gene-specific primers for human olfactory epithelium cDNA. −RT, reverse transcriptase was omitted. M, Marker sizes (base pairs) (C) Concentration-response ratios of (−)citronellal for rho-tag(39)-Olfr43 (filled circle), −LOC331758 (open circle), −OR1A1 (filled triangle), and −OR1A2 (open triangle) in HeLa-Cx43/CNGA2/G-alpha-olf cells. Similar results were obtained in three independent FLIPR experiments. Arrow, human threshold-concentration (0.3 μM).

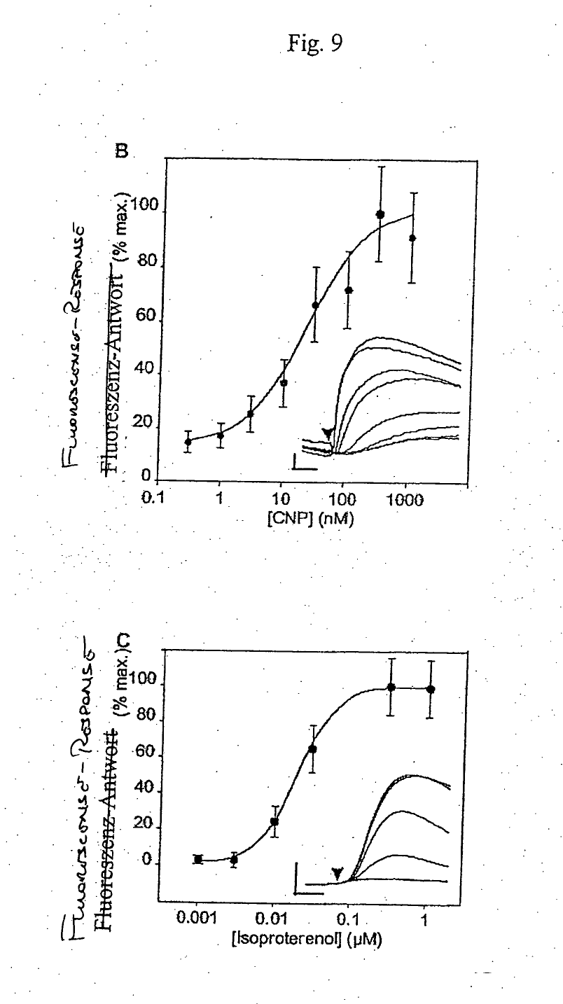

FIG. 9: Schematic depiction of the olfactory receptor-signalling pathway in HeLa-cells. The system consists of the receptor (A), the heterotrimeric G-protein (B), the adenyl cyclase (C), and the channel CNGA2 (D).

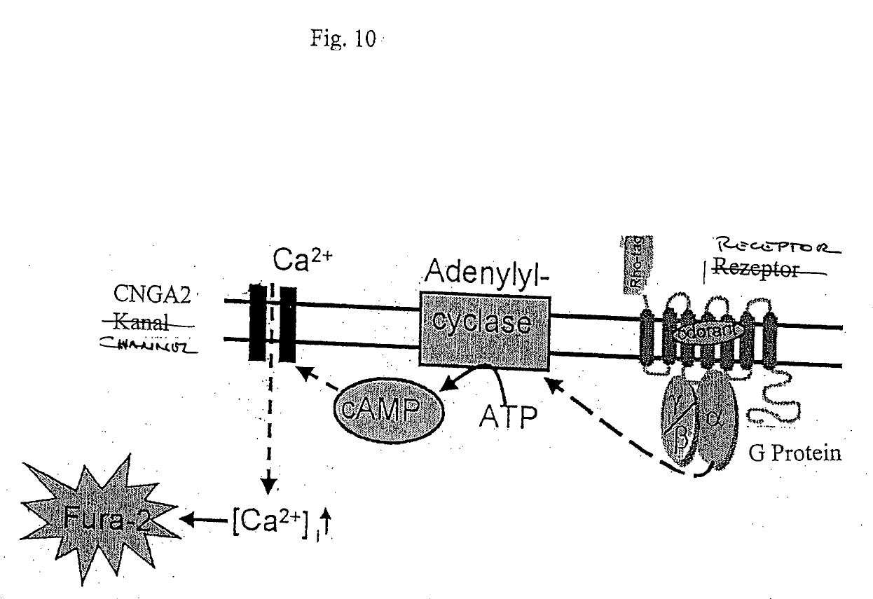

FIG. 10: General suitability of the cellular system for the characterisation of receptors that modify the intracellular concentration of cyclic nucleotides. (B) Particular guanylyl cyclase; (C) adrenergic receptor.

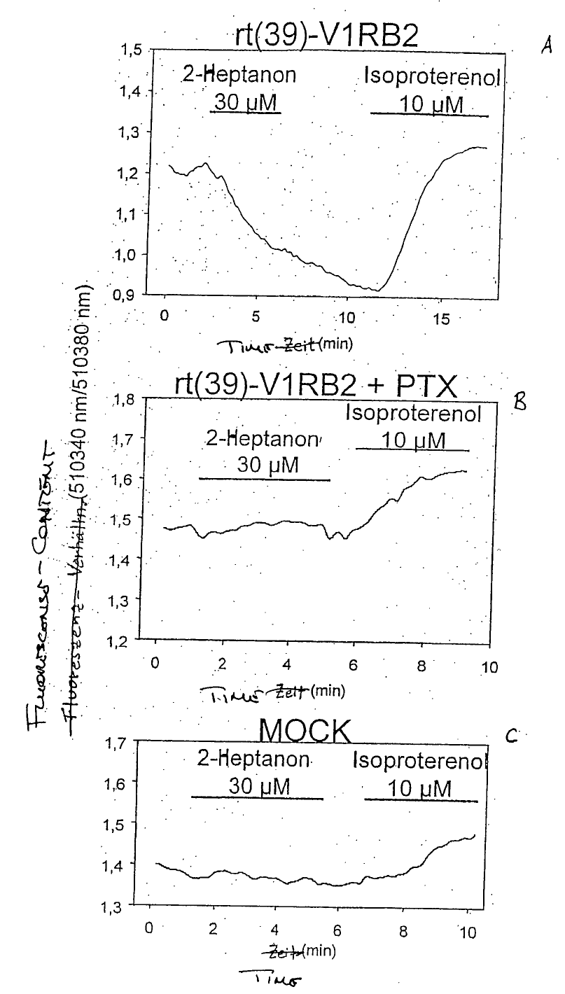

FIG. 11: General suitability of the cellular system for the characterisation of receptors that modify the intracellular concentration of cyclic nucleotides, using the example of a pheromone receptor rt(39)-V1R-b2 with 2-heptanone, (A). Negative control with pertussis-toxin (B), the toxin blocks the specific G protein G-alpha-I, (C) Empty control; all three experiments with isoproterol, which acts on the endogenous adrenergic receptors.

FIG. 12: RT-PCR products of a) HeLa/CNGA2 mRNA and b) HeLa/Olf cells using gene-specific primers for the human proteins Gαs and Gαolf. −RT=without reverse transcriptase, M, marker sizes (base pairs), c) Western blot analysis of HeLa/Cx43/CNGA2 cells (Gαs), and HeLa/Olf cells (Gαs and Gαolf). The anti-αs-antibody as used recognises both G-proteins Gαs and Gαolf having a size of 45 kDa. The 41 kDa-band is due to degradation or imprecise expression. The HeLa/Olf cells show an over-expression of the G-proteins.

### EXAMPLES

Molecular cloning—The inventors used rhodopsin-labelled rho-marker (20)-M4 chimeric mouse OR, but another subgroup of 93 ORs as reported by Krautwurst et al. (1998). CDNA that encoded for the bovine olfactory cyclic nucleotide-gating channel subunit CNGA2 (BTCAMPGC, X55010, Ludwig et al., 1990), was inserted into the vector pcDNA3.1/Zeo(+). In order to obtain the coding region of the olfactory human guanine nucleotide-binding protein alpha (hG-alpha-olf, GNAL: NM002071), the inventors first isolated human RNA from surgical olfactory epithelium biopsies with trizol (Gibco), then the mRNA with Micro-FastTrack 2.0 (Invitrogen), and synthesised first-strand cDNA using ImProm-II (Promega). The inventors PCR-amplified and subcloned hG-alpha-olf into the CMV promoter-driven expression cassette pi2-dk, based on plasmid pIRES2-EGFP (Clontech), which, nevertheless, lacked the IRES-EGFP part. The inventors PCR-amplified the full-length coding regions of mouse Olfr49 (1-C6, NM010991) and Olfr41 (17, NM010983) (Krautwurst et al., 1998) of their original plasmids, mouse Ors6 (NM020289, Malnic et al., 1999), Olfr43 (XM111129), LOC331758 (XM137710), MOR261-10 (NM146369), and MOR267-1 (NM146937) from mouse (C57BL/6J) genomic DNA, and the human OR1A1 (NM014565) and OR1A2 (NM012352) from human genomic DNA with Pfu (Promega) or PfuUltra (Stratagene). The amplicons were subcloned into pi2-dk(rt39). The cassette provides the first 39 amino acids of the bovine-rhodopsin (rho-tag(39)) as an N-terminal marker (Chandrashekar et al., 2000) for all full-length ORs. The identities of all subcloned amplicons were checked by sequencing (UKEHH, Hamburg).

Cell culture and transient DNA transfection—All cell culture media, ingredients and antibiotics were obtained from Invitrogen/Gibco, with the exception of G418 (Calbiochem) and puromycin (Sigma). HeLa cells were grown in Dulbecco's Modified Eagle Medium (DMEM) with 10% heat-inactivated foetal bovine serum (FBS), 2 mM L-glutamine, 100 U/ml penicillin, and 100 μg/ml streptomycin in a humidified atmosphere (37° C., 5% CO2).

Before transfection, the cells were seeded onto glass cover slides (VWR) for single cell Ca2+ imaging or black wall/clear bottom 96 well plates (Molecular Devices) for FLIPR, both coated with poly-D-lysine (10 μg/ml), and grown to a pre-confluent monolayer. The cells were transfected with DNA by using lipofection (PolyFect, Quiagen), and taken up into the experiments 40 hours after transfection.

Cloning and establishing of the expression plasmid for human G-alpha olf and the HeLa-Cx43/CNGA2/hG-alpha-olf (HeLa/Olf) cell line.

1) In order to obtain the coding region of the olfactory human guanine nucleotide-binding G protein-alpha (hG-alpha-olf, GNAL: accession-number NM002071), the inventors first isolated RNA from human surgical olfactory epithelium-biopsies with trizol (Gibco), then the mRNA was isolated with Micro-FastTrack 2.0 (Invitrogen), and first-strand cDNA was synthesized by using ImProm-II (Promega) reverse transcriptase (RT). The inventors PCR-amplified the full-length coding region of hG-alpha-olf using a Pfu DNA polymerase (Promega) and subcloned this into the CMV promoter-driven expression cassette pi2-dk, based on plasmid pIRES2-EGFP (Clontech), which, nevertheless, lacked the IRES-EGFP part.

2) Before transfection with hG-alpha-olf, HeLa-Cx43/CNGA2 cells were plated in 100 mm plates at a density of 1.6×10⁶ cells, and incubated over night. The cells were transfected with the expression plasmid hG-alpha-olf/pi2-dk which carried the coding regions for hG-alpha-olf through calcium phosphate precipitation. Then, HeLa/Olf cells were obtained through the selection of clonal populations that were resistant against 800 μg/ml G418 and responsive against (−)citronellal or isoproterenol. These were confirmed by RT-PCR or Western blot. Clonal lineages were held in standard DMEM, supplemented with puromycin (1 μg/ml), zeocin (100 μg/ml), and G418 (400 μg/ml). The inventors observed a stable expression of hG-alpha-olf until at least passage 10.

Single cell Ca2+ imaging—Single cell Ca2+ imaging was performed as described earlier (Bufe et al., 2002). The fluorescence of individual Fura-2-AM (Molecular Probes)-loaded cells were recorded at an emission wavelength of 515 nm following excitation with 340 nm and 380 nm, and calculated (F340/F380). Odorants, natriuretic peptide type C(CNP), forskolin, poly-D-lysin, probenecid, salts, buffers, and db-cAMP (dibutyryl cyclic adenosine monophosphate, membrane-permeable, used at 1 mM) were from Sigma. IBMX (3-isobutyl-1-methylxanthine), a blocker of the phosphodiesterase (100 μM, 30 min), was from Calbiochem. The forskolin pre-treatment (15 min) in single cell Ca2+ imaging-experiments always took place at 10 μM, nevertheless, was omitted in all experiments with HeLa-Cx43/CNGA2/hG-alpha-olf cells. Thapsigargin (BioTrend/Tocris; 1 μM, 30 min), a blocker of an intracellular Ca2+-ATPase, was used in order to increase cytoplasmatic levels of Ca2+, thus facilitating the OR-activated cAMP signalling via Ca2+-sensitive ACIII (Choi et al., 1992), or in order to avoid any receptor-mediated Ca2+ release from IP3-sensitive internal deposits (Thastrup et al., 1994). Bath-use of 1 mM EGTA (Sigma) was used, in order to confirm an influx of extracellular Ca2+ into the cells. All chemicals were of the highest purity available.

Fluorescence imaging plate reader (FLIPR) assay—The FLIPR (Molecular Devices) integrated an argon laser excitation source, a 96 well pipetter, and a detection system on the system, including a CCD (charged coupled device) imaging camera. The experiments were performed with FLUO-4 (4 μM, Molecular Probes)—loaded cells. A pre-treatment with forskolin (2.5 μM, 15 min) or IBMX (100 μM, 30 min) took place before the use of the agonists. The agonist-response amplitudes were determined from the peak-stimulated fluorescence of the solvent, control-subtracted, and base line-corrected measurements, and averaged over 4-5 wells which expressed the same receptor and received the same stimulus.

Data analysis—In the FLIPR screening experiments, responders were selected according to the criterion of ≧50% of the maximal increase of fluorescence within the first minute following odorant-use. Odorant-induced fluorescence amplitudes were normalised at the end of the experiment to the amplitude that was caused by 10 μM isoproterenol in the same well. The EC50 or IC50 values and curves were derived from fittings of the function f(x)=(a−d)/(1+(x/C)ˆnh)+d to the data by non-linear regression, with a=minimum, d=maximum, C=EC50 or IC50, and nH=Hill coefficient.

Immunocytochemistry—The plasma-membrane expression of CNGA2 or rho-tag-OR was detected by using the primary antibodies rabbit-anti-CNG2 (Alomone) or B6-30 (Margrave et al., 1986) that were directed against the C-terminus of CNGA2 or the N-terminal part of rhodopsin, respectively, in permeabilised or non-permeabilised cells. The cellular surface was visualised by detecting plasma-membrane glycoprotein with 20 μl/ml biotin-conjugated concanavalin A (Sigma), and staining with avidin-conjugated Texas Red (Molecular Probes), as described earlier (Bufe et al., 2002). Labelled CNGA2 or OR proteins were visualised by using Alexa-488-coupled secondary antibodies (goat-anti-rabbit, -anti-mouse, Molecular Probes), and confocal microscopy (Leica TCS SP2 Laser Scan).

cAMP test—HeLa-Cx43/CNGA2/rho-tag(39)-Olfr49 cells (10⁶ cells per well in 6-well plates) were used non-transfected, or were transfected with 1.5 μg rat G-alpha-olf or rat G-alpha-s DNA using PolyFect. After 40 hours, the cells were pre-incubated with IBMX (100 μM, 30 min) and exposed to (−)citronellal or isoproterenol (2 min). The pre-stimulation with forskolin was generally omitted. The cAMP levels were measured with the ¹²⁵I-labelled cAMP test system (Amersham) in threefold determinations. The values were normalised before the agonist-treatment against the level of cAMP. In order to estimate the mean cAMP concentration/cell, the inventors detected the average spheroid volume by measuring the diameter of 125 round cell-morphs using an Axioplan microscope (Zeiss), and the Metamorph Software (Universal Imaging Corp.). The μmol of cAMP, produced at the respective odorant EC50 were obtained from non-linear regression analysis.

Tissue and section preparation and in situ hybridisation—OEs were obtained from male adult C57BL/6J mice. Six week-old anesthetised mice were transcardially infunded with ice-cold PBS, and fixed with Bouin's solution (Sigma). The in situ hybridisation was performed at 65° C. with the respective digoxigenin-labelled sense and antisense riboprobes, produced with the DIG RNA labelling-mix (Roche) on serial coronal 14 μm cryosections.

Stable reconstitution of odorant/OR-induced cAMP/Ca2+ influx signalling transmission in HeLa cells—Initially, the inventors established the human HeLa-Cx43/CNGA2 cell line that stably expressed the olfactory homomeric CNGA2 channel as a reporter, in order to follow receptor-induced increases in intracellular cyclic nucleotides online. The inventors confirmed the expression of mRNA for the CNGA2 channel in HeLa-Cx43/CNGA2 cells by RT-PCR, and the plasma-membrane-expression of the CNGA2 protein by immunocytochemistry (FIG. 1). The homomeric CNGA2 channel has an EC50 for cGMP (3 μM) which is 20-fold lower as the one for cAMP (Finn et al., 1998). The cGMP signalling can be used in vivo by a subgroup of OSNs, including the olfactory particular guanylyl cyclase GC-D (Fulle et al., 1995). The inventors tested the possibility to follow an increase in cGMP by transfecting of the rat particular guanylyl cyclase type B (GC-B) into HeLa-Cx43/CNGA2 cells, for which the C type natriuretic peptide (CNP) is the known agonist (Lucas et al., 2000). In these cells, CNP in the presence of IBMX caused a GC-B-dependent and EGTA-sensitive Ca2+ influx, with an EC50 of 0.027 μM (+0.001 SD; n=2) (FIG. 1). A characterisation of HeLa-Cx43/CNGA2 cells by RT-PCR resulted in the expression of mRNA for G-alpha-s (FIG. 4) as well as for ACIII, VI, VII, and IX (FIG. 1). In these cells, in the presence of forskolin and increasing concentrations of the P-adrenergic receptor-(P-AR)-agonist isoproterenol, the inventors caused the cAMP production and Ca2+ influx with EC50 values of 0.028±0.004 μM and 0.026±0.008 μM, respectively. From this, the inventors concluded that in these cells the OR/odorant-induced cAMP signalling transmission and the Ca2+ influx by the CNGA2 channels can be established. Nevertheless, the work of Kurahashi and co-workers suggested that odorants themselves can also suppress the response of OSN by direct blocking of their CNG or voltage-gating channels (Kurahashi et al., 1994; Kawai, 1999). Thus, the inventors first tested increasing concentrations of several citronellic odorants and aliphatic aldehydes for inhibitory effects on the CNGA2-dependent Ca2+ influx into HeLa-Cx43/CNGA2 cells, which was directly activated by db-cAMP (FIG. 2). (−)Citronellal, which is one of the −400 key food odorants (Grosch, 2001), blocked the Ca2+ influx into these cells at concentrations of more than 3 μM, and with an IC50 of 131.7±33.9 μM (n=2) (FIG. 2). Octanal and heptanal at up to 100 μM, beta-citronellol at up to 3 mM, and citronellic acid at up to 300 μM did not block the db-cAMP-induced Ca2+ influx by CNGA2 (n=2). In order to examine the OR/odorant-induced cAMP signal transmission and the Ca2+ influx into HeLa-Cx43/CNGA2 cells, the inventors established the stable expression of rho-tag(39)-Olfr49 for which (−)citronellal was identified as the cognate odorant (Krautwurst et al., 1998). The inventors confirmed its expression on the plasma-membrane level by immunocytochemistry and confocal microscopy (FIG. 2). The diterpene forskolin (Seamon and Daly, 1986) can be used in order to directly activate types I-VIII of the 9 mammalian ACs (for a summary, see Smit and Iyengar, 1998). In the hands of the inventors, HeLa-Cx43/CNGA2 cells had to be pre-incubated with forskolin in order to measure a receptor-induced and CNGA2-dependent Ca2+ influx. This is possibly due to a suboptimal expression of G-alpha-s, since in Ca2+ imaging experiments both over-expressions of G-alpha-s (n=3) and G-alpha-olf (FIG. 5) could compensate for the pre-stimulation of ACs by forskolin. The inventors determined a pre-stimulating concentration of 10 μM forskolin, in order to allow for a subsequent maximal odorant-induced Ca2+ influx into HeLa-Cx43/CNGA2/rho-tag(39)-Olfr49 cells (FIG. 2). In these cells (−)citronellal at 10 μM, if pre-treated with 10 μM forskolin, stimulated an OR-dependent Ca2+ influx that could be completely antagonised by extracellular EGTA (FIG. 2). The quantitative comparison of the relative potencies of the agonists by determining of their EC50 values is a standard method in the GPCR and agonist-classification. The inventors thus used the FLIPR system, in order to establish concentration-response curves for cognate receptor-ligand pairs within concentration ranges, where no or only a slight blocking of CNGA2-dependent Ca2+ influx was observed. FLIPR experiments with HeLa-Cx43/CNGA2 cells that stably expressed rho-tag(39)-Olfr49 resulted in an EC50 value for (−)citronellal of 2.1±0.3 μM (n=3), compared to 4.0±2.2 μM (n=5) in cells with transiently transfected rho-tag(39)-Olfr49 (FIG. 3). In the rho-tag(39)-Olfr49 stable cell line (−)citronellal induced a cAMP production in a concentration-dependent manner (FIG. 3), with an EC50 value of 0.49 μM (±0.25 SD; n=2). In order to strengthen a correlation between the agonist-induced cAMP production, Ca2+ influx, and activation of the CNGA2 channel, the inventors calculated an average cAMP concentration of 48 μM or 68 μM within an individual HeLa-Cx43/CNGA2 cell at the EC50-concentrations for (−)citronellal or isoproterenol, respectively. This agonist/GPCR-induced average intracellular cAMP concentration could be well compared with the EC50 of 65 μM for cAMP on the homomeric olfactory CNGA2 channel that was obtained in electrophysiological experiments with inside-out patches of CNGA2-expressing HEK293 cells (Finn et al., 1998). Through testing of aliphatic aldehyde- and citronellal-related compounds on HeLa-Cx43/CNGA2/rho-tag(39)-Olfr49 cells the inventors obtained the odorant profile: (−)Citronellal>(+)citronellal>octanal>heptanal>>beta-citronellol, reflected by the EC50 values of 2.1±0.1 μM (n=4), 2.6±0.4 μM (n=3), 3.7±0.1 μM (n=3), 6.0±1.0 μM (n=3) and 32.8±2.7 μM (n=3), respectively (FIG. 3).

Stable reconstitution of odorant/OR-induced cAMP signalling transmission via G-alpha-olf-HeLa-Cx43/CNGA2 cells expressed the mRNA for the G protein subunit alpha-s, but not for G-alpha-olf (FIG. 4). In order to examine the efficiency of both G proteins in the OR signalling transmission, the inventors transfected the DNA for rG-alpha-olf or rG-alpha-s in HeLa-Cx43/CNGA2/rho-tag(39)-Olfr49 cells, and measured the (−)citronellal- and, via endogenous beta-AR, isoproterenol-induced cAMP production. In absence of forskolin or thapsigargin, the transfection with G-alpha-olf or G-alpha-s preferably increased the signalling transmission efficiency of rho-tag(39)-Olfr49 or endogenous beta-AR (FIG. 4), respectively. In order to improve the OR signalling transmission, the inventors thus established the stable expression of human G-alpha-olf in HeLa-Cx43/CNGA2 cells. (−)Citronellal caused an Ca2+ influx into HeLa-Cx43/CNGA2/hGaolf cells which expressed rho-tag(39)-Olfr49 (FIG. 5), and with a similar EC50 (2.2 μM) as in forskolin-pre-treated HeLa-Cx43/CNGA2/rho-tag(39)-Olfr49 cells (cf. FIG. 3). Amongst the four ACs that were expressed in the present HeLa-Cx43/CNGA2 cells (see FIG. 1), only the olfactory ACIII can be activated by Ca2+ in the presence of an active G protein alpha subunit (for a summary, see Smit and Iyengar, 1998). The inventors thus hypothesised that an increase in the intracellular Ca2+ concentration through a pre-treatment of the cells with thapsigargin will facilitate the cAMP signalling transmission of OR specifically via ACE. The inventors observed an odorant-induced Ca2+ influx into HeLa-Cx43/CNGA2/rho-tag(39)-Olfr49 cells without hG-alpha-olf only in those cases, where the cells were pre-treated with thapsigargin (FIG. 5). In HeLa-Cx43/CNGA2/hGaolf cells that expressed rho-tag(39)-Olfr49 and were pre-treated with thapsigargin, (−)citronellal caused a similar Ca2+ influx as in non-pre-treated cells (FIG. 5). The inventors then tested the three parameters ‘forskolin pre-treatment’, ‘thapsigargin pre-treatment’, and ‘hG-alpha-olf enrichment’ in cAMP tests with (−)citronellal-stimulated HeLa-Cx43/CNGA2/rho-tag(39)-Olfr49 cells. The (−)citronellal-induced cAMP production was significantly increased, and to the same extent in cells that were pre-treated with either forskolin or thapsigargin, compared to a stimulation with (−)citronellal in cells without pre-treatment (FIG. 5). The transfection with hG-alpha-olf led to the highest (−)citronellal-induced cAMP production. In these cells, thapsigargin reduced the (−)citronellal-induced cAMP production to a similar level, as was determined in thapsigargin- or forskolin-pre-treated cells without hG-alpha-olf (FIG. 5). In order to test the validity of the cellular system according to the invention for OR signalling transmission, the inventors used two other ORs, Olfr41 and Ors6, for which the cognate odorants heptanal and nonandione acid were each identified in Ca2+ imaging experiments with recombinant ORs, or isolated ORNs (Krautwurst et al., 1998; Malnic et al., 1999). In the hands of the inventors, heptanal at 10 μM triggered the Ca2+ influx into thapsigargin pre-treated HeLa-Cx43/CNGA2/hG-alpha-olf cells that expressed rho-tag(39)-Olfr41 (FIG. 5). In thapsigargin pre-treated HeLa-Cx43/CNGA2/hG-alpha-olf cells that expressed rho-tag(39)-Ors6, nonandione acid (FIG. 5), but, nevertheless, no other C8 carboxylic- or dicarboxylic acid or C9 carboxylic acid (n=2) induced a Ca2+ influx at 10 μM. Similar results were obtained with rho-tag(39)-Olfr41 and rho-tag(39)-Ors6 in HeLa-Cx43/CNGA2/hG-alpha-olf cells without thapsigargin-pre-treatment (n=2).

Screening and functional identification of mouse ORs for citronellic odorants—Through analysis of the “Human Genome Project” the inventors did not find well defined orthologs (−85% amino acid-identity) for Olfr49. The closest related ORs in the mouse or the human exhibited 54% or 56% amino acid-identity to Olfr49, respectively. Nevertheless, sensitivity-dependent hierarchic receptor codes for odours were proposed in a recent study (Hamana et al., 2003). The inventors thus started to identify other OR cognates for (−)citronellal with higher efficiency and specificity, compared to Olfr49. In order to avoid the combinatory burden of screening of hundreds of odorants versus −1000 ORs, the inventors screened about 10% of a total mouse OR genome, expressed in HeLa-Cx43/CNGA2/hG-alpha-olf cells. The inventors first tested both odorants at concentrations below their EC50 values for Olfr49, and (−)citronellal next to its odour threshold in the human (0.3 μM, Leffingwell, 2003). In FLIPR experiments, the inventors used 1 μM (−)citronellal or 10 μM beta-citronellol on HeLa-Cx43/CNGA2/hG-alpha-olf cell lines expressing 93 of a collection of 141 rho-tag(20)-M4 chimeric mouse ORs. The 93 ORs as tested here are exclusive of the 80 ORs that were tested in a prior study (Krautwurst et al., 1998). Nevertheless, the rho-tag(20)-M4 chimera of Olfr49 was included as a positive control (96-well coordinate A5) and responded to both odorants (FIG. 6), respectively. The inventors furthermore identified two OR chimeras (3%, 96 well coordinates A1 and F2) that responded to 1 μM (−)citronellal, wherein F2 also reacted to 10 μM beta-citronellol (FIG. 6). 20 OR chimeras (22%), including rho-tag(20)-M4-Olfr49 (A5) responded to 10 μM beta-citronellol (FIG. 6), wherein H1 showed the strongest normalised response. An increase of the concentration of (−)citronellal from 1 μM to 3 μM increased the number of responding OR chimeras to 40 (43%), including the OR chimeras at the coordinates A1, A5 and F2. The inventors observed a similar increase in the percentage of responding ORs in three other screening-experiments with a subgroup of 67 OR chimeras which partially overlapped with the 93 ORs as shown here, when the concentration of (−)citronellal was increased from 1 μM (9%, n=1) to 3 μM (22% and 59%). The OR chimeras A5 and F2 were included in these experiments, and in all cases responded to both concentrations of (−)citronellal. None of the 93 OR chimeras responded to citronellic acid up to 10 μM (n=2). Blasting of the TMII-VII sequences of the three newly identified responders from the 96 well coordinates A1, F2 and H1 against the GenBank resulted in the corresponding mouse OR gene, Olfr43, mOR267-1, and mOR261-10, respectively. These ORs show 35-41% amino acid-identity among each other, and with Olfr49. The A1, F2 and H1 rho-tag(20)-M4-chimeras as well as their respective rho-tag(39)-full length ORs selectively responded to 1 μM (−)citronellal (Olfr43, Olfr49, mOR267-1) or 10 μM beta-citronellol (Olfr49, mOR267-1, mOR261-10) in single cell Ca2+ imaging experiments, when expressed in thapsigargin-pre-treated HeLa-Cx43/CNGA2/hG-alpha-olf cells (n=3).

Spatial gene expression in the OE of OR for citronellic odorants—Until now, the Olfr43 differs from all other ORs that were identified by the inventors as responsive against citronellic odorants identified. Olfr43 showed a specificity for (−)citronellal at 1 μM above 10 μM of other citronellic odorants, such as, for example, beta-citronellol (see FIG. 7) or citronellic acid (n=2). The inventors thus hypothesised that the specific function of Olfr43 reflects a topographic expression in the OE which differs from the expression of the other ORs. In in situ RNA hybridisation-experiments, the inventors examined the gene expression of Olfr41, Olfr43, Olfr49, MOR261-10, MOR267-1, and Ors6 in OSN of the mouse OE. Ors6 and Olfr41, which responded to the aliphatic odorants nonandione acid and heptanal, each marked the most dorso-medial and ventro-lateral expression patterns in coronal section of mouse OE (FIG. 7). In between, the (−)citronellal and beta-citronellol responders Olfr49, MOR261-10 and MOR267-1 showed an overlapping zonal expression, with a laterally shifted expression of Olfr43 (FIG. 7). The functional identification of a human ortholog OR for mouse Olfr43. A characterisation of syntenic OR clusters of the human chromosome 17p13.3 and mouse chromosome 11B3-B5 led to the identification of orthologous ORs (Glusman et al., 2000; Lapidot et al., 2001). OR1A1 and OR1A2 were found as the two closest human homologs to Olfr43, sharing 84% and 77% amino acid-identity with Olfr43 and their closest mouse-homolog, LOC331758 (99%), respectively (FIG. 8, Lapidot et al., 2001). RT-PCR experiments showed an mRNA expression of OR1A1 and OR1A2 in human olfactory epithelium (FIG. 8). When expressed in HeLa-Cx43/CNGA2/hG-alpha-olf cells, rho-tag(39)-Olfr43, −LOC331758, —OR1A1, and −OR1A2 showed similar EC50 values for (−)citronellal, 2.1±0.2 μM (n=3), 3.2±0.8 μM (n=3), 2.2±0.4 μM (n=3), and 2.4±0.7 μM (n=2), respectively (FIG. 8).

### REFERENCES

Araneda R. C., Kini A. D. and Firestein S. (2000) The molecular receptive range of an odorant receptor. Nat Neurosci, 3, 1248-55.
Bakalyar H. A. and Reed R. R. (1990) Identification of a specialized adenylyl cyclase that may mediate odorant detection. Science 250, 1403-6.
Baker H., Cummings D. M., Munger S. D., Margolis J. W., Franzen L., Reed R. R. and Margolis F. L. (1999) Targeted deletion of a cyclic nucleotide-gated channel subunit (OCNC1): biochemical and morphological consequences in adult mice. J Neurosci, 19, 9313-21.
Belluscio L, Gold G. H., Nemes A. and Axel R. (1998) Mice deficient in G(olf) are anosmic. Neuron, 20, 69-81.
Bozza J., Feinstein P., Zheng C. and Mombaerts P. (2002) Odorant receptor expression defines functional units in the mouse olfactory system. J Neurosci, 22, 3033-43.
Brunet L. J., Gold G. H. and Ngai J. (1996) General anosmia caused by a targeted disruption of the mouse olfactory cyclic nucleotide-gated cation channel. Neuron 7, 681-93.
Buck L. and Axel R. (1991) A novel multigene family may encode odorant receptors: a molecular basis for odor recognition. Cell, 65, 175-87.
Bufe B., Hofmann T., Krautwurst D., Raguse J. D. and Meyerhof W. (2002) The human TAS2R16 receptor mediates bitter taste in response to beta-glucopyranosides. Nat Genet 32, 397-40).
Chandrashekar J., Mueller K. L, Hoon M. A., Adler E., Feng L, Guo W., Zuker C. S. and Ryba N.J. (2000) T2Rs function as bitter taste receptors. Cell, 100, 703-11.
Chess A., Simon I., Cedar H. and Axel R. (1994) Allelic inactivation regulates olfactory receptor gene expression. Cell, 78, 823-34.
Choi E. J., Xia Z. and Storm D. R. (1992) Stimulation of the type III olfactory adenylyl cyclase by calcium and calmodulin. Biochemistry, 31, 6492-8.
Crider J. Y. and Sharif N. A. (2002) Adenylyl cyclase activity mediated by beta-adrenoceptors in immortalized human trabecular meshwork and non-pigmented ciliary epithelial cells. J Ocul Pharmacol Ther, 18, 221-30.
Dhallan R. S., Yau K. W., Schrader K. A. and Reed R. R. (1990) Primary structure and functional expression of a cyclic nucleotide-activated channel from olfactory neurons. Nature, 347, 184-7.
Duchamp-Viret P., Duchamp A. and Chaput M. A. (2000) Peripheral odor coding in the rat and frog: quality and intensity specification. J Neurosci, 20, 2383-90.
Dzeja C., Hagen V., Kaupp U. B. and Frings S. (1999) Ca2+ permeation in cyclic nucleotide-gated channels. Embo J, 18, 131-44.
Finn J. T., Krautwurst D., Schroeder J. E., Chen J. Y., Reed R. R. and Yau K. W. (1998) Functional co-assembly among subunits of cyclic-nucleotide-activated, nonselective cation channels, and across species from nematode to human. Biophys J, 74, 1333-45.
Firestein S., Darrow B. and Shepherd G. M. (1991) Activation of the sensory current in salamander olfactory receptor neurons depends on a G protein-mediated cAMP second messenger system. Neuron, 6, 825-35.
Fulle, H. J., Vassar, R., Foster. D. C., Yang, R. B., Axel, R. and Garbers, D. L. (1995) A receptor guanylyl cyclase expressed specifically in olfactory sensory neurons. Proc Natl Acad Sci USA 92, 3571-5.
Gaillard I., Rouquier S., Pin J. P., Mollard P., Richard S., Barnabe C., Demaille J. and Giorgi D. (2002) A single olfactory receptor specifically binds a set of odorant molecules. Eur J Neurosci, 15, 409-18.
Glusman G., Sosinsky A, Ben-Asher E., Avidan N., Sonkin D., Bahar A., Rosenthal A., Clifton S., Roe B., Ferraz C., Demaille J. and Lancet D. (2000) Sequence, structure, and evolution of a complete human olfactory receptor gene cluster. Genomics, 63, 227-45.
Gold G. H. (1999) Controversial issues in vertebrate olfactory transduction. Annu Rev Physiol, 61, 857-71.
Grosch W. (2001) Evaluation of the key odorants of foods by dilution experiments, aroma models and omission. Chem Senses, 26, 533-45.
Hamana H., Hirono J., Kizumi M. and Sato T. (2003) Sensitivity-dependent Hierarchical Receptor Codes for Odors. Chem Senses. 28, 87-104.
Hargrave P. A., Adamus G., Arendt A., McDowell J. H., Wang J., Szaby A., Curtis D. and Jackson R. W. (1986) Rhodopsin's amino terminus is a principal antigenic site. Exp Eye Res. 42, 363-73.
Jones D. T., Masters S. B., Bourne H. R. and Reed R. R. (1990) Biochemical characterization of three stimulatory GTP-binding proteins. The large and small forms of Gs and the olfactory-specific G-protein.Golf. J Biol Chem, 265, 2671-6.
Jones D. T. and Reed R. R. (1989) Golf: an olfactory neuron specific-G protein involved in odorant signal transduction. Science, 244, 790-5.
Juilfs D. M., Fulle H. J., Zhao A. Z., Houslay M. D., Garbers D. L. and Beavo J. A. (1997) A subset of olfactory neurons that selectively express cGMP-stimulated phosphodiesterase (PDE2) and guanylyl cyclase-D define a unique olfactory signal transduction pathway. Proc Natl Acad Sci USA 94, 3388-95.
Kajiya K., Inaki K., Tanaka M., Haga J., Kataoka H. and Touhara K. (2001) Molecular bases of odor discrimination: Reconstitution of olfactory receptors that recognize overlapping sets of odorants. J Neurosci, 21, 6018-25.
Kawai F. (1999) Odorants suppress T- and L-type Ca2+ currents in olfactory receptor cells by shifting their inactivation curves to a negative voltage [In Process Citation]. Neurosci Res, 35, 253-63.
Krautwurst D., Yau K. W. and Reed R. R. (1998) Identification of ligands for olfactory receptors by functional expression of a receptor library. Cell, 95, 917-26.
Kurahashi J., Lowe G. and Gold G. H. (1994) Suppression of odorant responses by odorants in olfactory receptor cells. Science, 265, 118-20.
Lapidot X., Pilpel Y., Gilad Y., Falcovitz A., Sharon D., Haaf J. and Lancet D. (2001) Mouse-human orthology relationships in an olfactory receptor gene cluster. Genomics, 71, 296-306.
Leffingwell J. C. (2003) Chirality & Odour Perception—Acyclic Terpenoid Odorants. http://www.leffingwell.com/chirality/acyclic_terpenoid.htm.
Liu H. Y., Wenzel-Seifert K. and Seifert R. (2001) The olfactory G protein G(alphaolf) possesses a lower GDP-affinity and deactivates more rapidly than G(salphashort): consequences for receptor-coupling and adenylyl cyclase activation. J Neurochem, 78, 325-38.
Lucas K. A., Pitari G. M., Kazerounian S., Ruiz-Stewart I., Park J., Schulz S., Chepenik K. P. and Waldman S. A. (2000) Guanylyl cyclases and signaling by cyclic GMP. Pharmacol Rev, 52, 375-414.
Ludwig J., Margalit J., Eismann E., Lancet D. and Kaupp U. B. (1990) Primary structure of cAMP-gated channel from bovine olfactory epithelium. FEBS Lett, 270, 24-9.
Malvl. and Shepherd G. M. (2000) Functional mosaic organization of mouse olfactory receptor neurons. Proc Natl Acad Sci USA, 97, 12869-74.
Malnic B., Hirono J., Sato T. and Buck L. B. (1999) Combinatorial receptor codes for odors. Cell, 96, 713-23.
Meyer M. R., Angele A., Kremmer E., Kaupp U. B. and Muller F. (2000) A cGMP-signaling pathway in a subset of olfactory sensory neurons. Proc Natl Acad Sci USA, 97, 10595-600.
Mombaerts P. (1999) Molecular biology of odorant receptors in vertebrates. Annu Rev Neurosci, 22, 487-509.
Nakamura T. and Gold G. H. (1987) A cyclic nucleotide-gated conductance in olfactory receptor cilia. Nature, 325, 442-4.
Omura M., Sekine H., Shimizu J., Kataoka H. and Touhara K. (2003) In situ Ca2+ imaging of odor responses in a coronal olfactory epithelium slice. Neuroreport. 14, 1123-7.
Reed R. R. (1992) signaling pathways in odorant detection. Neuron, 8, 205-9.
Sanchez-Montanes M. A. and Pearce T. C. (2002) Why do olfactory neurons have unspecific receptive fields? Biosystems, 67, 229-38.
Sato T., Hirono J., Tonoike M. and Takebayashi M. (1994) Tuning specificities to aliphatic odorants in mouse olfactory receptor neurons and their local distribution. J Neurophysiol, 72, 2980-9.
Scott J. W. and Brierley T. (1999) A functional map in rat olfactory epithelium. Chem Senses, 24, 679-90.
Scott J. W., Brierley T. and Schmidt F. H. (2000) Chemical determinants of the rat electro-olfactogram. J Neurosci, 20, 4721-31.
Seamon K. B. and Daly J. W. (1986) Forskolin: its biological and chemical properties. Adv Cyclic Nucleotide protein Phosphorylation Res, 20, 1-150.
Sicard G. and Holley A. (1984) Receptor cell responses to odorants: similarities and differences among odorants. Brain Res, 292, 283-96.
Smit M J. and lyengar R. (1998) Mammalian adenylyl cyclases. Adv Second Messenger Phosphoprotein Res, 32, 1-21.
Thastrup O., Dawson A. P., Scharff O., Foder B., Cullen P. J., Drobak B. K., Bjerrum P. J., Christensen S. B. and Hanley M. R. (1994) Thapsigargin, a novel molecular probe for studying intracellular calcium release and storage. 1989. Agents Actions, 43, 187-93.
Touhara K. (2002) Odor discrimination by G protein-coupled olfactory receptors. Microsc Res Tech, 58, 135-41.
Touhara K., Sengoku S., Inaki K., Tsuboi A., Hirono J., Sato T., Sakano H. and Haga T. (1999) Functional identification and reconstitution of an odorant receptor in single olfactory neurons. Proc Natl Acad Sci USA, 96, 4040-5.
Wetzel C. H., Oles M., Wellerdieck C., Kuczkowiak M., Gisselmann G. and Hatt H. (1999) Specificity and sensitivity of a human olfactory receptor functionally expressed in human embryonic kidney 293 cells and Xenopus Laevis oocytes. J Neurosci 19, 7426-33.
Wong S. T., Trinh K., Hacker B., Chan G. C., Lowe G., Gaggar A., Xia Z., Gold G. H. and Storm D. R. (2000) Disruption of the type III adenylyl cyclase gene leads to peripheral and behavioral anosmia in transgenic mice. Neuron, 27, 487-97.
Young J. M. and Trask B. J. (2002) The sense of smell: Genomics of vertebrate odorant receptors. Hum Mol Genet, 11, 1153-60.
Zhang X. and Firestein S. (2002) The olfactory receptor gene superfamily of the mouse. Nat Neurosci, 5, 124-33.
Zhao H., Ivic L., Otaki J. M., Hashimoto M., Mikoshiba K. and Firestein S. (1998) Functional expression of a mammalian odorant receptor. Science, 279, 237-42.
Zozulya S., Echeverri F. and Nguyen J. (2001) The human olfactory receptor repertoire. Genome Biol, 2, RESEARCH0018.

1. A recombinant cellular system, comprising an animal host cell, comprising the following recombinant proteins
a recombinant specific G protein-coupled receptor, and
a recombinant CNGA2 Ca2+ permeable channel.

2. The recombinant cellular system according to claim 1, further comprising a recombinant protein selected from the group of connexins.

3. The recombinant cellular system according to claim 1, wherein the recombinant specific G protein-coupled receptor is selected from type A guanylyl-cyclases and type G guanylyl-cyclases.

4. The recombinant cellular system according to claim 1 further comprising a cyclase that is harmonised with the specific G protein-coupled receptor.

5. The recombinant cellular system according to claim 1 wherein the recombinant specific G protein-coupled receptor is selected from: pheromone receptors, hormone receptors, and the olfactory receptors.

6. The recombinant cellular system according to claim 1 further comprising a recombinant G-protein that is harmonised with the specific G protein-coupled receptor.

7. The recombinant cellular system according to claim 1 wherein the animal host cell is selected from murine cell lines and human cell lines.

8. The recombinant cellular system according to claim 1, wherein the cellular system comprises a potential recombinant specific G protein-coupled receptor.

9. The recombinant cellular system according to claim 7, selected from the group of cellular systems comprising: HeLa-Cx43/CNGA2/Olfr49; HeLa-Cx43/CNGA2/G-alpha-olf; HeLa-Cx43/CNGA2/G-alpha-olf/Olfr 49; HeLa-Cx43/CNGA2/G-alpha-olf/Olfr41; HeLa-Cx43/CNGA2/G-alpha-olf/Olfr 6 and HeLa-Cx43/CNGA2/G-alpha-olf/OR1A1.

10. The recombinant cellular system according to claim 1, wherein the recombinant proteins are present stably.

11. The recombinant cellular system HeLa-Cx43/CNGA2/G-alpha-olf, as deposited on Apr. 20, 2004 at the DSMZ—Deutsche Sammlung von Mikroorganismen and Zellkulturen GmbH in Mascheroder Weg 1b, D-38124 Braunschweig with the deposit number DSM ACC2649.

12. A method for producing a recombinant cellular system, comprising the steps of:
providing of an animal host cell,
introducing a recombinant specific G protein-coupled receptor or a potential recombinant specific G protein-coupled receptor, and
introducing the recombinant CNGA2 Ca2+ permeable channel.

13. The method according to claim 12, further comprising the step of:
introducing of a recombinant protein from the group of the connexins.

14. The method according to claim 12, further comprising the step of:
introducing of a cyclase that is harmonised with the specific G protein-coupled receptor.

15. The method according to claim 12, further comprising the step of:
introducing of a recombinant G-protein that is harmonised with the specific G protein-coupled receptor.

16. The method according to claim 12, wherein the introducing method step is selected from:
(Ca2+-phosphate-)transfection, lipofection or electroporation,
optionally followed by the step of integration into the genome with the aid of a recombinase or antibiotic-selection cloning, or the step of transduction.

17. The method for identifying receptor activating substances, comprising the method steps of
providing a recombinant cellular system according to claim 1,
contacting of the cellular system with a potential G protein-coupled receptor activating substance, and
measuring the activation or inhibition of the Ca2+ influx into the cellular system.

18. The method according to claim 17, wherein the potential G protein-coupled receptor inducing substance is selected from odorants, pheromones, and hormones.

19. The method according to claim 17, wherein the measuring of the Ca2+ influx into the cell includes: loading of the cell with Fura-2-AM or Fluo-4-AM, and measuring of the emission-wavelength at 515 nm.

20. The method according to claim 17, wherein the cellular system is pre-treated with an enhancer.

21. A method for producing a pharmaceutical composition, comprising the steps of:
performing a method according to claim 17, and
formulating of the obtained G protein-coupled receptor inducing substance with auxiliary agents and additives.

22. A method for identifying of G protein-coupled receptors, comprising the steps of:
providing a recombinant cellular system according to claim 8,
contacting of the cellular system with a receptor-activating substance or presumably receptor-activating substance, and
measuring the activation or inhibition of the Ca2+ influx into the cell.

23. The method according to claim 17, wherein the method is performed in a high-throughput-environment.

24. (canceled)

25. (canceled)
